(19) United States
(12) Patent Application Publication
(10) Pub. No.: US 2023/0025256 A1
(43) Pub. Date: Jan. 26, 2023

(54) PROTEIN BLOCKING ASSEMBLY AND METHODS OF MAKING AND USING

(71) Applicants: SIMON FRIEDMAN, KANSAS CITY, MO (US); SWETHA CHINTALA, KANSAS CITY, MO (US); MAYANK SHARMA, KANSAS CITY, MO (US)

(72) Inventors: SIMON FRIEDMAN, KANSAS CITY, MO (US); SWETHA CHINTALA, KANSAS CITY, MO (US); MAYANK SHARMA, KANSAS CITY, MO (US)

(21) Appl. No.: 17/782,717

(22) PCT Filed: Dec. 3, 2020
(86) PCT No.: PCT/US2020/063041

§ 371 Date: Jun. 6, 2022

Related U.S. Application Data
(60) Provisional application No. 62/943,470, filed on Dec. 4, 2019.

(51) Int. Cl. C07K 14/605; C07K 14/62; C07K 14/705

(52) U.S. Cl. CPC C07K 14/605; C07K 14/62; C07K 14/70503

(57) ABSTRACT

Certain aspects of the present invention are directed to a method for making a protein blocking assembly reversibly linking a therapeutic protein to a blocking group via a linker moiety. Additional aspects of the invention are directed to the protein blocking assembly, and to methods of administering the protein blocking assembly to a subject, where the protein is cleaved from the protein blocking assembly.

### CROSS-REFERENCE TO RELATED APPLICATIONS

This application is based on and claims priority to U.S. Provisional Application Ser. No. 62/943,470 filed on Dec. 4, 2019, which is hereby incorporated herein by reference.

### STATEMENT REGARDING FEDERALLY SPONSORED RESEARCH OR DEVELOPMENT

This invention was made with government support under Contract No. DP3DK106921 awarded by the National Institutes of Health. The government has certain rights in the invention.

### FIELD OF THE INVENTION

Aspects of the present invention relate generally to therapeutic proteins. More specifically, certain aspects of the invention relate to assemblies for blocking protein self-association, and methods of making and using the assemblies.

### BACKGROUND OF THE INVENTION

Proteins (and peptides) are classes of pharmaceuticals that are of increasing importance, and that command a growing proportion of newly identified therapeutics. Part of this expansion is due to the diversity of structures that are readily available, as well as the ease of identifying protein-based binding partners for specific therapeutic targets. One of the main problems with proteins is their ability to self-associate. This creates self-associated species with limited efficacy or altered pharmacological properties. FIG. 1 depicts one type of such protein self-association.

Two examples of this phenomenon occur with glucagon and insulin. Glucagon is a signaling molecule that can be provided exogenously to stimulate blood glucose increases in diabetics. It does this by binding to the glucagon receptor. Glucagon has limited solubility at neutral pH, but is readily dissolved in acidic solutions. Once in solution however, glucagon molecules rapidly associate with other glucagon molecules to form semi-regular structures called fibrils. These thin, string-like collections of glucagon are no longer able to bind to the glucagon receptor and therefore lose pharmacological activity. Because of this, it is challenging to have conveniently usable formulations of glucagon. It also prevents use of glucagon in the development of an artificial pancreas due to the fibrils clogging the catheter, in addition to the other limitations discussed above.

Insulin is a related signaling molecule that also binds to a receptor, and induces the uptake of blood glucose. Insulin is soluble at neutral pH, but over time can self-associate to also form fibrils. These fibrils, like in the case of glucagon, do not retain the ability of monomeric insulin molecules to bind to their target receptor. Thus, insulin bioactivity degrades over time due to fibril formation. In addition to fibril formation, insulin can self-associate to form hexamers. This hexamer formation can be encouraged by including zinc in formulations of insulin. Hexamers are resistant to fibril formation, but have their own problems. Specifically, they are large compared to monomeric insulin and so absorb much more slowly than does monomeric insulin, thus slowing their desired physiological response (blood glucose reduction).

The field has attempted to deal with the problem of protein self-association by altering the primary structure of proteins such as glucagon and insulin, in order to remove the potential sites of interaction that drive the association. This has worked to a degree, but inherently requires the creation of a non-natural protein. This introduces a potential source of toxicity, as it introduces a non-native protein into the body. These issues with insulin and glucagon are illustrative of a general problem: the self-association of proteins, leading to an altering of their pharmacological properties.

### BRIEF SUMMARY OF THE INVENTION

Certain aspects of the present invention are directed to a method for reversibly preventing a protein from interacting with a second protein by forming a protein blocking assembly. The assembly may be produced by forming a first bond between a linking moiety and a protein and forming a second bond between the linking moiety and blocking group, such at least one of the first and second bonds is cleavable.

The structure of the blocking group prevents interaction between the protein of the protein blocking assembly and a second protein due to steric or static interference. In certain embodiments, this inhibits formation of protein-protein complexes, such as hexamers and fibrils. The stearic interference may be caused by the blocking group's size, shape or combinations thereof, and the electrostatic interference may be caused by the blocking group's charge, partial charge or combinations thereof. In certain embodiments, the blocking group comprises a peptide, lipid, small molecule, nucleic acid, saccharide or combinations thereof. In embodiments where the blocking group is a peptide, the peptide may have a sequence of CEₙ, where n=1-5, preferably n=5. In certain embodiments, the peptide is from 1 to 100 amino acids in length.

In certain embodiments, the protein is insulin, glucagon, immunoglobulin, their derivatives or analogs, and combinations thereof. The protein bond between the protein and the linking moiety may be via a natural or added side chain functional group of the protein. Suitable protein side chains for forming a cleavable bond with the linking moiety include amine, alcohol, carboxylic acid, guandinium, amide, thiol and combinations thereof.

In certain embodiments, the bond between the protein and linking moiety, or the bond between the linking moiety and blocking group, is cleavable by chemical effector or enzyme, such as a chemical effector or enzyme endogenous to the body of the human or animal subject to which the protein blocking assembly is administered. In certain embodiments, the bond or bonds are cleavable by hydrolysis by esterases, hydrolysis by peptidases, hydrolysis by phosphatases, hydrolysis by other enzymes, reduction, oxidation, or combinations thereof.

In certain embodiments, the linker moiety comprises activated pyridyl dithio ethanol (PDE), which may be activated, such as by carbonyldiimidazole (CDI).

In certain embodiments, the cleavable bonds may include esters, amides, carbamates, carbonates, phospho-esters, phosphor-amides, di-sulfides, ethers, ketals, aminals, acetals, sulfonamides, imines, hydrazones, or combinations thereof.

Other aspects of the invention are directed to the protein blocking assembly produced by any of the methods disclosed, using any of the constituents disclosed herein. Other aspects of the invention are directed to administering any such protein blocking assembly to a human or animal in need of the protein. Once administered, the protein is cleaved from the protein blocking assembly. Such cleavage may occur by a chemical or biochemical processes endogenous to the human or animal.

Other aspects of the invention are directed to a protein blocking assembly that includes a protein, reversibly linked to a blocking group by a linking moiety, where the structure of the blocking group prevents interaction with a second protein due to steric or static interference.

Additional aspects of the invention, together with the advantages and novel features appurtenant thereto, will be set forth in part in the description that follows, and in part will become apparent to those skilled in the art upon examination of the following, or may be learned from the practice of the invention. The objects and advantages of the invention may be realized and attained by means of the instrumentalities and combinations particularly pointed out in the appended claims.

### BRIEF DESCRIPTION OF THE DRAWINGS

FIG. 1 depicts the self-association of therapeutic proteins without the blocking groups of the present disclosure, creating inactive fibrils or complexes with altered activity.

FIG. 2 depicts protein blocking assemblies of the present invention and a method for cleaving the protein from the assembly.

FIG. 3 depicts two methods for converting a modified glucagon to native, active protein.

FIG. 4 depicts the formation of activated PDE from PDE and carbonyldiimidazole (CDI).

FIG. 5 depicts the bonding of activated PDE to glucagon.

FIGS. 6A and 6B show the HPLC trace purity analysis and ESI-MS, respectively, confirming the correct species for the activated PDE-glucagon assembly of FIG. 5.

FIG. 7 depicts the bonding of PDE-glucagon to a peptide blocking group CE.

FIGS. 8A and 8B show the HPLC trace purity analysis and ESI-MS, respectively, confirming the correct species for the glucagon-PDE-CE assembly of FIG. 7.

FIG. 9 depicts the bonding of PDE-glucagon to a peptide blocking group CE₂.

FIGS. 10A and 10B show the HPLC trace purity analysis and ESI-MS, respectively, confirming the correct species for the glucagon-PDE-CE₂ assembly of FIG. 9.

FIG. 11 depicts the bonding of PDE-glucagon to a peptide blocking group CE₃.

FIGS. 12A and 12B show the HPLC trace purity analysis and ESI-MS, respectively, confirming the correct species for the glucagon-PDE-CE₃ assembly of FIG. 11.

FIG. 13 depicts the bonding of PDE-glucagon to a peptide blocking group CE₄.

FIGS. 14A and 14B show the HPLC trace purity analysis and ESI-MS, respectively, confirming the correct species for the glucagon-PDE-CE₄ assembly of FIG. 13.

FIG. 15 depicts the bonding of PDE-glucagon to a peptide blocking group CE₅.

FIGS. 16A and 16B show the HPLC trace purity analysis and ESI-MS, respectively, confirming the correct species for the glucagon-PDE-CE₅ assembly of FIG. 15.

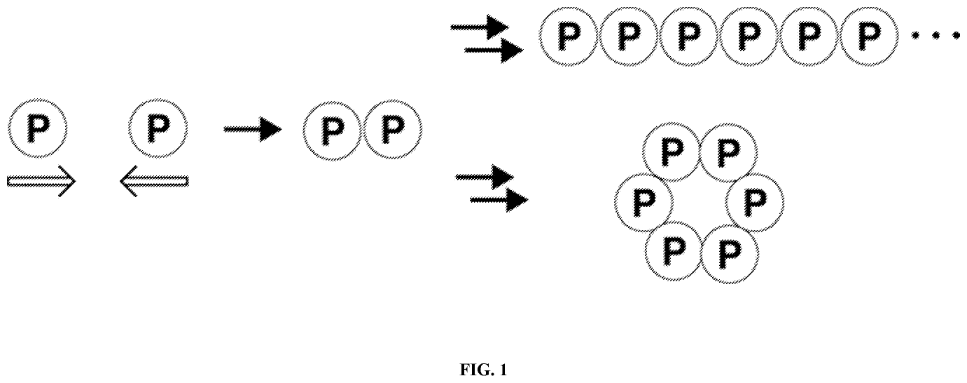

### DETAILED DESCRIPTION OF PREFERRED EMBODIMENT

The present application provides a method for preventing protein self-association without changing the primary structure of the therapeutic protein. In certain aspects, the method involves forming a protein blocking assembly by reversibly linking a therapeutic protein to a blocking group via a linker moiety. The linker moiety creates one or more reversible bonds between the protein and blocking group. The resulting protein blocking assembly presents a stable form of the protein that inhibits formation of fibrils, hexamers or other protein-protein complexes and structures. When administered to a subject, one or more of the bonds is cleaved to release the native, active protein.

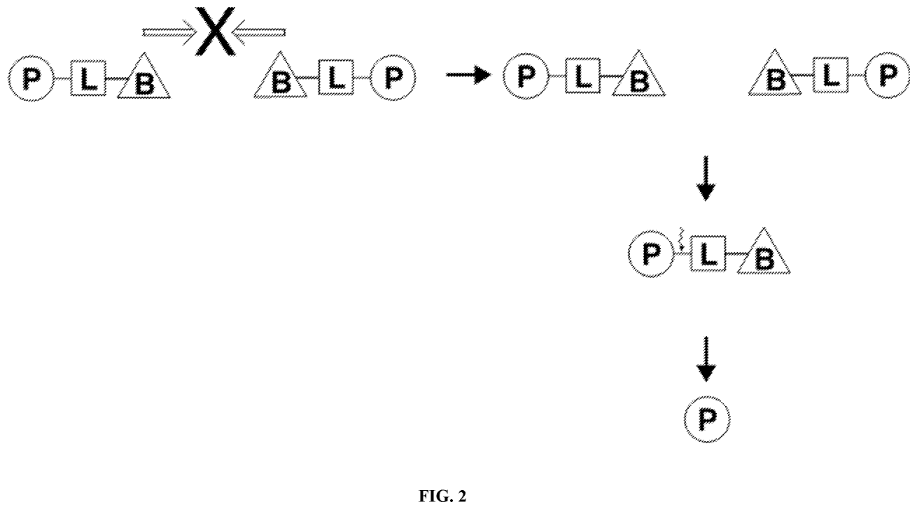

One method of assembly consistent with the present disclosure is illustrated in FIG. 2. A blocking group (B) is reversibly linked to protein (P) via a cleavable linker moiety (L), preventing self-association of the protein. The linker moiety, may first be linked to the protein and then linked to the blocking group, or the linker moiety may first be linked to the blocking group and then linked to the protein. Any or all of the protein, linking moiety and blocking groups may be functionalized to facilitate formation of the bonds between the protein, linking moiety and blocking moiety.

The resulting protein blocking assembly is administered to a subject in a pharmaceutically acceptable formulation. The protein blocking assembly is preferably stable in the formulation. However, after administration, the blocking group is cleaved from the protein in the body to reveal the native, active protein, as illustrated in FIG. 2. In certain embodiments, the blocking group may be removed by endogenous chemical or biochemical processes. For example, the blocking group may be removed by a chemical effector or an enzyme, preferably a chemical effector or enzyme present in the body of the subject, more preferably by a chemical effector or enzyme naturally occurring in the body of the subject. A chemical effector is any chemical that can effect cleavage of the bond connecting the blocking group and/or protein to the linking moiety. In other embodiments, the blocking group may be linked to the protein via a photocleavable linker moiety, and the blocking group may be removed by application of a light source with a wavelength matched to the photocleavable linker moiety.

The aim of the blocking group is to interfere with the contact of one protein molecule with another. The blocking group is preferably selected to interfere with the point of contact of one protein with another. The blocking may be brought about by different properties of the blocking group, including steric factors (size and/or shape) and electrostatic factors (charges and/or partial charges) that cause repulsion between protein blocking assemblies.

It is expected that the nature of the blocking group will be different for different proteins. Suitable blocking groups include peptides, lipids, small molecules, nucleic acids, saccharides or combinations thereof. The blocking group may be charged or contain charged sub-groups that will result in charged protein blocking assemblies that will repel each other to prevent self-association.

In certain embodiments, the blocking groups are small molecules, so that there is no need for further biodegradation of the blocking group once it is cleaved from the protein. The blocking groups can be easily cleared from the body by natural pathways after cleavage. The blocking group does not comprise a long polymer chain and is not a polymer backbone to which multiple drugs are cross-linked. In certain embodiments, the molecular weight of the blocking group is less than 5000, or from 20 to 5000. In certain embodiments, the ratio of protein to blocking group in the protein blocking assembly is greater than 2:1 (wt:wt), from 2:1 to 100:1, or from 2:1 to 50:1.

The blocking group preferably contains, or is modified to contain, a functional group suitable for bonding to the protein via a linker moiety. Suitable functional groups on the blocking group include amines, alcohols, carboxylic acids, guanidinium, amide, thiols or a combination thereof. Other suitable functional groups may include vinylsulfone, alkyne, azide, maleimide, isothiocyanate, isocyanate, imidate, alpha-halo-amide, Michael acceptor, hydrazide, oxyamine, hydrazine, alkyl, aryl, alkenyl, alkynyl, cyano, nitro, azido, heterocycles and combinations thereof or with the previously listed functional groups. Terminal groups may be added to protect and/or modify the charge of blocking group. Such modifications are considered to be within the scope of the peptides and other blocking groups disclosed herein.

Peptides are particularly promising as blocking groups. A wide range of peptide functionalities, with highly variable physical properties, can easily be generated and assessed. Peptides may be substituted or unsubstituted. Suitable peptides are no longer than 100 amino acids, preferably from 1 to 50 amino acids. Amino acids that can be incorporated in to the peptide include the standard naturally occurring 20 amino acids, as well as unnatural amino acids that contain the D configuration. The peptides may be predominantly cationic, predominantly anionic, polar but uncharged, hydrophobic or a mixture of all these properties. In certain aspects of the invention, a thiol-containing peptide, such as a peptide containing a cysteine, may be used. In certain embodiments the peptide incudes cysteine and from 1-5 glutamic acid molecules. Other exemplary blocking groups include lipids, saccharides, and nucleic acids.

In certain aspects, the blocking group is bioresorbable. As used here, the term “bioresorbable” refers to a group whose degradative products, or the group itself, are metabolized in vivo or excreted from the body via natural pathways. In general, by “bioresorbable,” it is meant that the group will be broken down and absorbed within the human body, for example, by a cell or tissue.

In certain aspects, the blocking group is biocompatible. As used herein the term “biocompatible” means that the group will not cause substantial tissue irritation or necrosis when administered. Preferably, the group is approved for use in the body by the Food and Drug Administration.

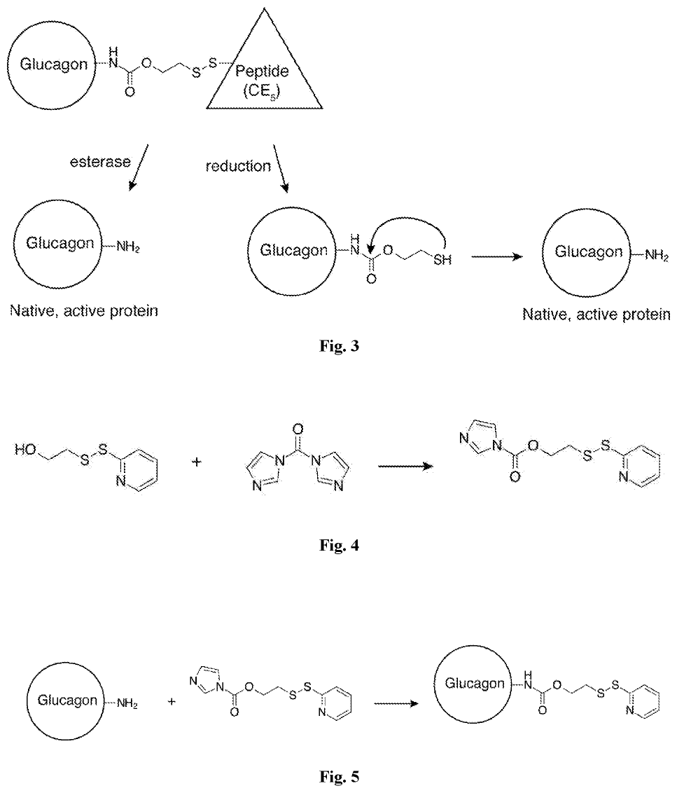

The blocking group may be linked to the protein via a linker moiety. The linker moiety that joins the blocking group to the protein preferably forms one or more cleavable bonds between the blocking group and/or the protein. In certain embodiments, the linking moiety forms a first bond with the protein and a second bond with the blocking group to form the protein blocking assembly. At least one of the first and second bonds is cleavable. Preferably, the bond between the protein and the linking moiety is cleavable, and a single cleavage is required to release the protein in its native form, as shown in FIG. 2 and the bottom left reaction of FIG. 3. However, the bond between the blocking group and the linking moiety may also be cleavable. In such embodiments, the blocking group may be cleaved from the assembly, followed by cleavage of the bond between the protein and the linking moiety, as shown on in the bottom right reaction of FIG. 3.

As used herein, a cleavable or reversible bond is a bond that can be cleaved by chemical, enzymatic, photolytic or other mechanism to release the linked blocking group and/or protein, preferably without alteration of the native form of the blocking group or protein. Preferably the cleavable bonds are sensitive to endogenous chemical and/or enzymatic reactions, i.e. reactions that naturally take place in the body of a subject to which the protein blocking assembly is administered. Examples of such reactions include hydrolysis by esterases, hydrolysis by peptidases, hydrolysis by phosphatases, hydrolysis by other enzymes, reduction, oxidation and combinations thereof. Alternatively, the blocking group may be linked to the protein via a photocleavable linker moiety, and the blocking group may be removed by application of a light source with a wavelength matched to the photocleavable linker moiety. In certain other embodiments, other chemical or enzymatic reactants can be introduced to the subject to cleave one or more of the bonds of the protein blocking assembly, either before, simultaneously with, or after administration of the protein blocking assembly.

The linker moiety preferably comprises chemical groups that produce bonds sensitive to chemical, enzymatic and/or photolytic reactions. Bonds formed between the linker moiety and the peptide and/or blocking group consistent with the present invention include but are not limited to esters, amides, carbamates, carbonates, phospho-esters, phosphor-amides, di-sulfides, ethers, ketals, aminals, acetals, sulfonamides, imines, and/or hydrazones and as well as other groups known to be useful in forming prodrugs. Chemical groups may be added to the linker moiety, peptide and/or blocking group to facilitate formation of desired bonds. Exemplary functional groups in the linking moiety that can be used to create the cleavable bonds with the protein and/or blocking group, include amino, carboxyl, thiol, phosphate, and/or alcohol. Suitable chemical groups and bonds will depend in part on the physiological characteristics of the portion of the body into which the protein blocking assembly will be administered. Suitable photocleavable linkers are disclosed in U.S. Pat. Pub. 2020/0147215 and U.S. Pat. No. 10,159,735, which are incorporated herein for such disclosure.

The linker moiety is preferably bioresorbable and biocompatible. In general, the linker moiety can include any agent that may be linked to the peptide and blocking group and which, upon exposure to physiological conditions, chemical effectors, enzymes, and/or light, releases the therapeutic peptide in functional form (or a suitable prodrug form). In certain embodiments, the linker moiety length may be 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, or 50 atoms (e.g., carbons) long.

In certain embodiments, the linker moiety may be comprised of carbon, nitrogen, oxygen, sulfur, phosphorous atoms, or combinations thereof. For example, the linker may be an alkyl or contain ether, ester, and/or amines groups. In certain embodiments, the linker moiety is pyridyl dithio ethanol (PDE), preferably activated PDE. The PDE may be activated by a condensing agent, for example carbonyldiimidazole, as depicted in FIG. 4, and then reacted with the protein, as depicted in FIG. 5.

The linker moiety preferably is linked to the protein via a chemical side chain functional group on the protein. The protein may contain a suitable functional group, or a functional group may be added to the peptide to facilitate the bond. Suitable functional groups include amines, alcohols, carboxylic acids, guanidinium, amide, and thiol. Functional groups that may be added to the protein include carboxylic halide, vinylsulfone, alkyne, azide, maleimide, isothiocyanate, isocyanate, imidate, alpha-halo-amide, Michael acceptor, hydrazide, oxyamine, and/or hydrazine.

The protein may be any therapeutic protein useful in the treatment or prevention of a disease or condition. A “therapeutic protein” as used herein refers to any peptide or protein that alters the physiology of a patient. The term “therapeutic” may be used interchangeably herein or in the art with the terms “biologically active” and “pharmaceutically active” and includes analogs and derivatives of a therapeutic protein. Thus, the “therapeutic protein” that is cleaved form the protein blocking assembly may be a drug, drug precursor, prodrug, or modified drug that is not fully active or available until converted in vivo to its therapeutically active or available form. The protein may be naturally occurring or synthetic.

The protein blocking assembly is particularly useful with therapeutic proteins that are vulnerable to self-association. Representative non-limiting classes of proteins useful in the present invention include those falling into the following therapeutic categories: dACE-inhibitors; anti-anginal drugs; anti-arrhythmias; anti-asthmatics; anti-cholesterolemics; anti-convulsants; anti-depressants; anti-diarrhea preparations; anti-histamines; anti-hypertensive drugs; anti-infectives; anti-inflammatory agents; anti-lipid agents; anti-manics; anti-nauseants; anti-stroke agents; anti-thyroid preparations; anti-tumor drugs; anti-tussives; anti-uricemic drugs; anti-viral agents; acne drugs; alkaloids; amino acid preparations; anabolic drugs; analgesics; anesthetics; angiogenesis inhibitors; antacids; anti-arthritics; antibiotics; anticoagulants; antiemetics; antiobesity drugs; antiparasitics; antipsychotics; antipyretics; antispasmodics; antithrombotic drugs; anxiolytic agents; appetite stimulants; appetite suppressants; beta blocking agents; bronchodilators; cardiovascular agents; cerebral dilators; chelating agents; cholecystokinin antagonists; chemotherapeutic agents; cognition activators; contraceptives; coronary dilators; cough suppressants; decongestants; deodorants; dermatological agents; diabetes agents; diuretics; emollients; enzymes; erythropoietic drugs; expectorants; fertility agents; fungicides; gastrointestinal agents; growth regulators; hormone replacement agents; hyperglycemic agents; hypnotics; hypoglycemic agents; migraine treatments; mineral supplements; mucolytics; narcotics; neuroleptics; neuromuscular drugs; peripheral vasodilators; polypeptides; prostaglandins; psychotropics; renin inhibitors; respiratory stimulants; stimulants; sympatholytics; thyroid preparations; tranquilizers; uterine relaxants; vaginal preparations; vasoconstrictors; vasodilators; vertigo agents; and wound healing agents.

In one aspect, the protein is of the type described in Bossard et al., U.S. Patent Application No. 2011/0166063 and Ekwuribe, U.S. Pat. No. 6,858,580, which are incorporated by reference herein with respect to such disclosures. Preferred therapeutic peptides and proteins are selected from the group consisting of insulin; glucagon; calcitonin; gastrin; parathyroid hormones; angiotensin; growth hormones; secretin; luteotropic hormones (prolactin); thyrotropic hormones; melanocyte-stimulating hormones; thyroid-stimulating hormones (thyrotropin); luteinizing-hormone-stimulating hormones; vasopressin; oxytocin; protirelin; peptide hormones such as corticotropin; growth-hormone-stimulating factor (somatostatin); G-CSG, erythropoietin; EGF; physiologically active proteins, such as interferons and interleukins; superoxide dismutase and derivatives thereof; enzymes such as urokinases and lysozymes; and analogues or derivatives thereof. In another aspect, the therapeutic protein is selected from the group consisting of human growth hormone, bovine growth hormone, growth hormone-releasing hormone, an interferon, interleukin-1, interleukin-II, insulin, calcitonin, erythropoietin, atrial natriuretic factor, an antigen, a monoclonal antibody, somatostatin, adrenocorticotropin, gonadotropin releasing hormone, oxytocin, vasopressin, analogues, or derivatives thereof.

In another aspect, the therapeutic protein is an anti-diabetic agent already in the clinical practice or in the pipeline of development. The anti-diabetic drug molecules are broadly categorized herein as insulin/insulin analogs and non-insulin anti-diabetic drugs.

In certain embodiments, the protein is insulin, glucagon, or immunoglobulin. One preferred therapeutic peptide is glucagon (or an analog or derivative thereof). Another preferred therapeutic peptide insulin (or an analog or derivative thereof). As used herein, the term insulin embraces analogues or derivatives thereof. Exemplary insulin compounds are described in Foger et al., U.S. Published Patent No. 2011/0144010, which is incorporated by reference with respect to such disclosures. In another aspect, the therapeutic peptide is insulin (or an analog or derivative thereof) in its hexameric form, typically in the presence of zinc.

Certain aspects of the invention are directed to the protein blocking assembly produced using any of the methods, blocking groups, proteins and/or linking moieties discussed herein. Additional aspects of the invention are directed to methods for using any such protein blocking assemblies.

The protein blocking assembly of the present invention may be administered to a subject in need of the protein. After administration to the subject, the protein blocking assembly is cleaved to release the protein, allowing the protein to perform its intended function. Preferably, the protein is in its native state after being cleaved. In certain embodiments, the protein blocking assembly comprises a first bond between the linking moiety and the protein and second bond between the linking moiety and the blocking group, and one or both of the bonds is cleaved after administration. Preferably, at least the first bond between the linking moiety and the protein is cleaved.

The bonds of the protein blocking assembly may be cleaved by any mechanisms discussed herein. For example, the bonds can be cleaved by chemical, enzymatic, photolytic or other mechanism to release the linked blocking group and/or protein, preferably without alteration of the native form of the blocking group or protein. Preferably the cleavable bonds are sensitive to endogenous chemical and/or enzymatic reactions, i.e. reactions that naturally take place in the body of a subject to which the protein blocking assembly is administered. Examples of such reactions include hydrolysis by esterases, hydrolysis by peptidases, hydrolysis by phosphatases, hydrolysis by other enzymes, reduction, oxidation and combinations thereof. Alternatively, the blocking group may be linked to the protein via a photocleavable linker moiety, and the blocking group may be removed by application of a light source with a wavelength matched to the photocleavable linker moiety. In certain other embodiments, other chemical or enzymatic reactants can be introduced to the subject to cleave one or more of the bonds of the protein blocking assembly, either before, simultaneously with, or after administration of the protein blocking assembly.

The subject of the present invention is preferably an animal (for example, warm-blooded mammal) and may be either a human or a non-human animal. Exemplary non-human animals include but are not limited to non-human primates, rodents, farm animals (for example, cattle, horses, pigs, goats, and sheep) and pets (for example, dogs, cats, ferrets, and rodents). The patient is typically a mammal. The term “mammal” refers to organisms from the taxonomy class “mammalian,” including but not limited to humans, chimpanzees, apes, orangutans, monkeys, rats, mice, cats, dogs, cows, horses, etc.

The method of administration will depend on the therapeutic protein used. Suitable methods of administration include cutaneous, subcutaneous, intravenous or intramuscular injection. The protein blocking assembly can also be delivered nasally, transbuccally, sub-lingually or via similar administration routes. In certain embodiments, the protein blocking assembly can be administered in a manner similar to that used for the native peptide. Is it further contemplated that the protein-blocking group can be administered through an artificial pancreas system.

The protein blocking assembly is typically administered to the target site of the subject using a “cannula” or “needle” that can be a part of a drug delivery device, e.g., a syringe, a gun drug delivery device, or any medical device suitable for the application of a drug to a targeted organ or anatomic region. The cannula or needle of the protein blocking assembly is designed to cause minimal physical and psychological trauma to the subject.

Cannulas or needles include tubes that may be made from materials, such as for example, polyurethane, polyurea, polyether(amide), PEBA, thermoplastic elastomeric olefin, copolyester, and styrenic thermoplastic elastomer, steel, aluminum, stainless steel, titanium, metal alloys with high non-ferrous metal content and a low relative proportion of iron, carbon fiber, glass fiber, plastics, ceramics or combinations thereof. The cannula or needle may optionally include one or more tapered regions. The cannula or needle may be beveled. The cannula or needle may also have a tip style vital for accurate treatment of the patient depending on the site for implantation. Examples of tip styles include, for example, Trephine, Cournand, Veress, Huber, Seldinger, Chiba, Francine, Bias, Crawford, deflected tips, Hustead, Lancet, or Tuohey. The cannula or needle may also be non-coring and have a sheath covering it to avoid unwanted needle sticks. The dimensions of the hollow cannula or needle, among other things, will depend on the site for injection.

The protein blocking assembly of the present invention is formulated in a pharmaceutically acceptable carrier. It will be appreciated to those skilled in the art that the carrier may optionally contain inactive materials such as saline, buffering agents and pH adjusting agents such as potassium bicarbonate, potassium carbonate, potassium hydroxide, sodium acetate, sodium borate, sodium bicarbonate, sodium carbonate, sodium hydroxide or sodium phosphate; degradation/release modifiers; drug release adjusting agents; emulsifiers; preservatives such as benzalkonium chloride, chlorobutanol, phenylmercuric acetate and phenylmercuric nitrate, sodium bisulfate, sodium bisulfite, sodium thiosulfate, thimerosal, methylparaben, polyvinyl alcohol and phenylethyl alcohol; solubility adjusting agents; stabilizers; and/or cohesion modifiers. If the assembly is to be injected the spinal area, the carrier may comprise sterile preservative free material.

Upon administration, the protein should be rapidly released from the protein-blocking group. For example, the protein may be cleaved from the protein blocking assembly by chemical effector or enzyme. In certain embodiments, the bond between linker moiety and the blocking group and/or protein is cleavable by hydrolysis by esterases, hydrolysis by peptidases, hydrolysis by phosphatases, hydrolysis by other enzymes, reduction, and/or oxidation.

Certain aspects of the present invention are illustrated by the following non-limiting example.

### EXAMPLE 1

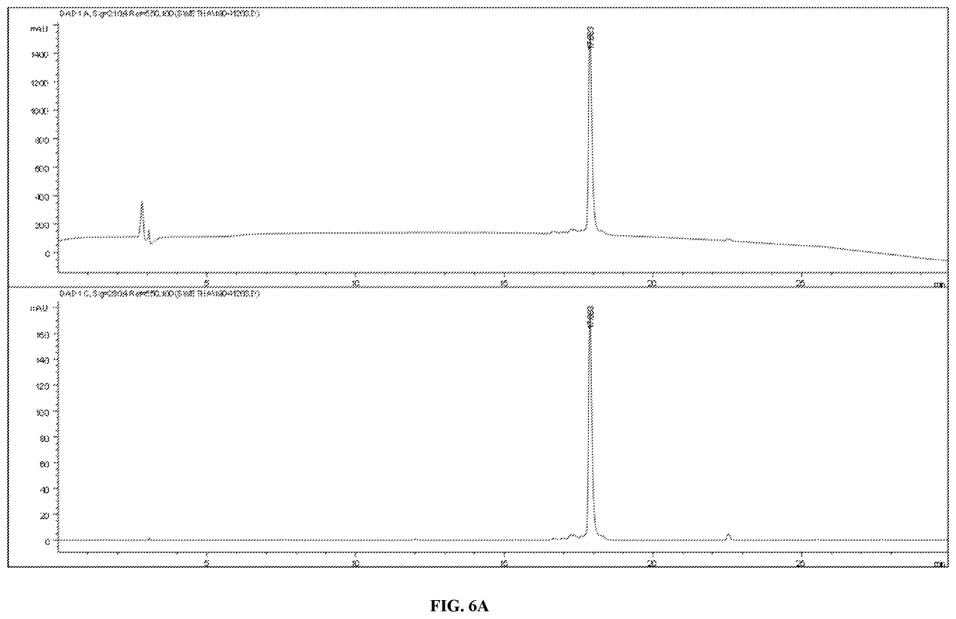

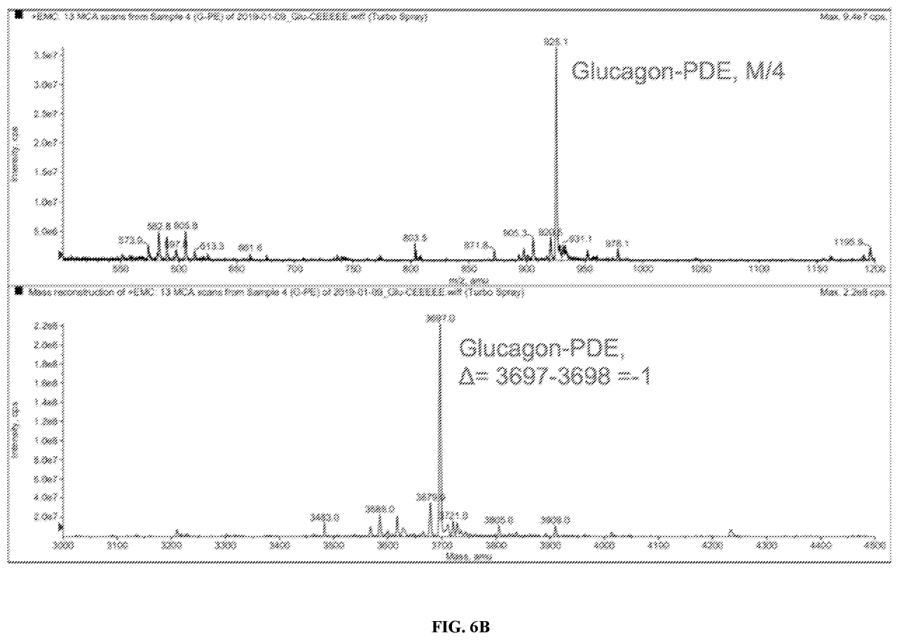

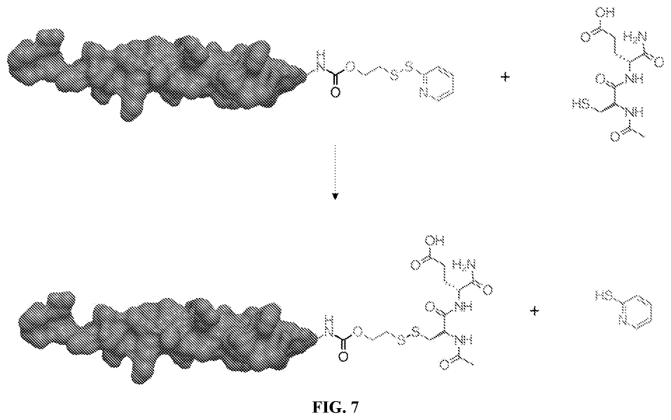

Exemplary embodiments of the present disclosure are shown in FIGS. 4 to 16A and 16B. In these embodiments, the protein is glucagon, linked to a peptide blocking group via a pyridyl dithio ethanol (PDE) linker moiety. More specifically, as depicted in FIG. 4, the reagent PDE is reacted with carbonyldiimidazole (CDI) to create activated PDE as the linker moiety. As depicted in FIG. 5, the activated PDE is then reacted with glucagon to form the carbamate linked species shown. HPLC and MS traces confirming formation of the correct species are shown in FIGS. 6A and 6B.

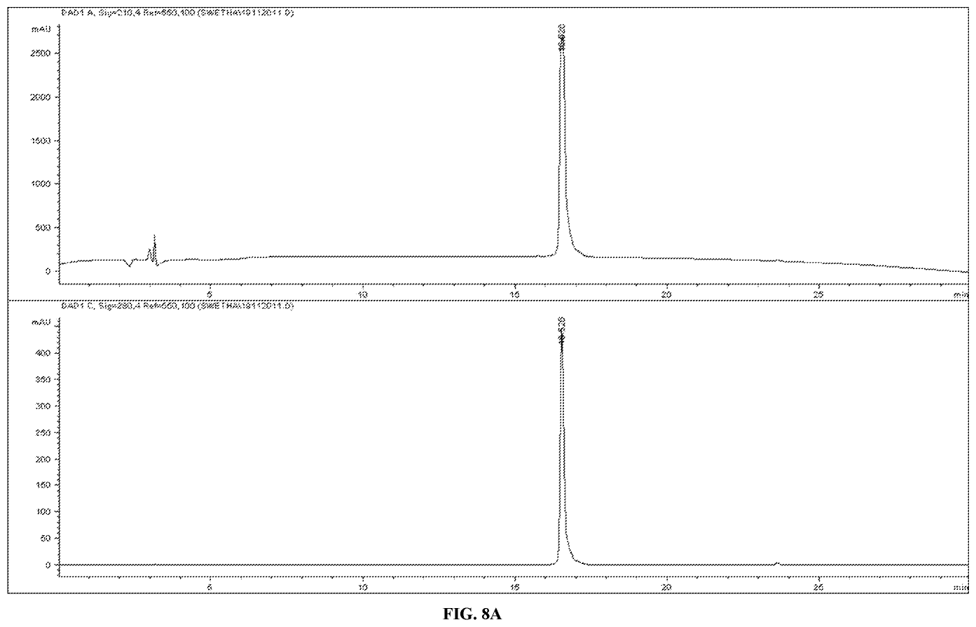

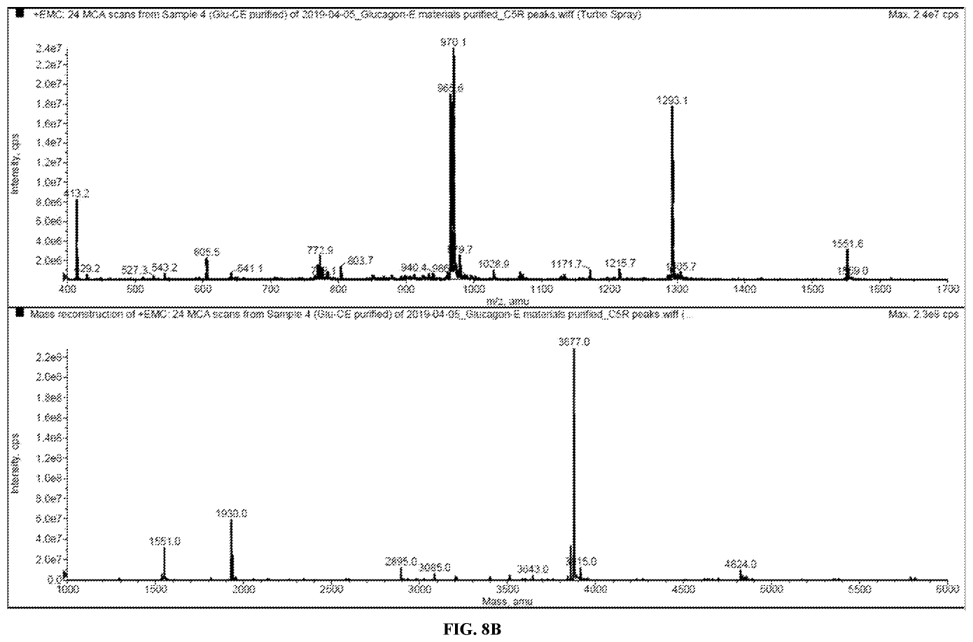

This intermediate protein-linker complex is reacted with a peptide that contains a thiol group, as shown in FIGS. 7 to 16A &B. The peptide acts as the blocking group, meant to interfere with glucagon's interaction with other glucagon molecules. In the exemplary embodiments, the peptide has a sequence of CEₙ, with C being a cysteine that contains a thiol group, E being glutamic acid, and n being a number from 1-5. The thiol can then react with the modified glucagon to make the final species of protein blocking assembly, as shown in FIGS. 7, 9, 11, 13 and 15, for peptides CE₁, CE₂, CE₃, CE₄, and CE₅, respectively. FIGS. 8A and B, 10A and B, 12A and B, 14A and B and 16A and B show the HPLC and MS traces confirming formation of the correct species of protein blocking assembly. Although 1-5 glutamic acid molecules are used in the examples, additional glutamic acid groups, up to 10, 15, 20, or more, could be used.

The final protein blocking assembly includes glucagon (protein P), linked to a blocking group (the peptide B), by a carbamate bond formed between the protein and the linker moiety (L). As illustrated in FIG. 3, the carbamate bond is subject to esterase cleavage when administered to a subject, which would release native glucagon. In addition, the disulfide bond between the blocking group and the linker moiety (L) may be subject to reductive cleavage, followed by release of glucagon through a ‘self-immolative’ process.

It will be understood by one of ordinary skill in the art that the schemes as shown in the figures can be readily varied as described herein. For example, any peptide sequence with a native or added thiol can be used as the blocking group. Further, in all of these examples, the peptides were synthesized as the C terminal carboxamide, and the N terminal has been acetylated. This was done to prevent the additional charges that these terminal groups would introduce but is not required of the overall method.

Exemplary processes used to form the protein blocking assemblies of FIGS. 7-16 follow:

Materials

1,1′-Carbonyldiimidazole (CDI), Human Glucagon from AmbioPharm, Rink amide resin (0.7-0.9 meq/g, 70-90 mesh), Fmoc-L-glutamic acid γ-tert-butyl ester hydrate, Fmoc-S-trityl-L-cysteine, 1-Hydroxybenzotriazole hydrate, N,N-Diisopropylethylamine, N-Methyl-2-pyrrolidone (DIPEA), Tri fluoroacetic acid 1-Methyl-2-pyrrolidinone (NMP), HOBt hydrate, HATU, 2,4,6-Trimethylpyridine (Collidine), Triisopropylsilane, Acetonitrile, HPLC grade water, dimethyl sulfoxide, methylene chloride, piperidine, 1,8-Diazabicyclo(5.4.0)undec-7-ene (DBU), acetic anhydride

An Ocean Optics USB-2000 fiber optic spectrophotometer with DT-Mini-B lamp source was used for UV/visible spectrophotometric analysis. Analytical HPLC analysis was performed on a Hewlett Packard Agilent 1050 HPLC with a diode array detector, using a 150×3.2 mm 5 μm Nucleosil C18 column (Supelco Analytical from Sigma Aldrich). Mass spectrometry was performed using a Q-Trap 3200 (electrospray ionization) attached to a Shimadzu UFLC system. Analyst v.1.4.2 software was used for the LC-MS analyses. Masses greater than 1700 were obtained by deconvoluting primary spectra of the different charge states using the Bioanalyst software. Preparative purification of compounds was done on a Thermo Scientific Ultimate 3000 HPLC with an automated fraction collector, using a Phenomenex 250×21.2 mm 10 μm C18 column.

Methods

Solid Phase Peptide Synthesis:

Peptides used to synthesis of modified glucagon materials were made using solid phase peptide synthesis. Resin was swelled in DCM and then washed with NMP. Five times excess of amino acid was activated with HOBt and DIPEA and added to resin. Coupling step was performed for 3 hours followed by 5 times washes with NMP and capping. 10% acetic anhydride, 5% DIPEA in NMP was used for capping. Capping was followed by NMP washes and Fmoc deprotection step. 20% piperidine and 2% DBU in NMP was used for Fmoc deprotection and deprotection was done thrice for completion. Fmoc-Cysteine was activated using HOBt/HATU/collidine in 1:1 DMF/DCM. Cysteine amino acid activation was performed in situ. After all the couplings were completed, resin was washed with DCM and cleaved using cleavage mixture (80:10:5:5=TFA:water:TIS:phenol). Cleavage mixture containing peptide was dried under N₂, and dried crude peptide was washed using cold diethyl ether. This crude peptide was then purified using reversed phase HPLC, characterized using HPLC and LCMS.

PDE Activation

Turning to FIG. 4, in an exemplary embodiment, 1.44 mmoles (0.269 grams) of PDE in 7.2 mL was activated with 1.44 mmoles (0.233 grams) of 1,1′-Carbonyldiimidazole (CDI). Mixture was kept at 40° C. for one hour.

Glucagon-PDE Synthesis

Turning to FIG. 5, in an exemplary embodiment, 71.8 μmoles (0.25 grams) of glucagon was dissolved in 60 ml of DMSO and the activated PDE mixture was added to glucagon in DMSO. Final reaction volume was adjusted to 71.8 mL. This reaction mixture was kept at 40° C. for 14 days.

The reaction was analyzed for the formation of the product using reversed phase HPLC. Reversed phase HPLC (flow rate 0.4 mL/min, runtime: 30 minutes with 5 minutes post-run), solvent A (0.1% TFA in H₂O), solvent B (0.1% TFA in acetonitrile), gradient 0% B to 100% B over 28 minutes, 100% B to 0% B over 2 minutes, post-run 0% B for 5 minutes, C18 Supelco Nucleosil column (5 μm, 150×3.2 mm). Glucagon-PDE isomer 2 eluted at 17.883 minutes. Glucagon-PDE was purified using semi preparative reversed phase HPLC. Reversed phase HPLC (flow rate 2 mL/min, runtime: 60 minutes with 10 minutes pre-run), solvent A (0.1% TFA in H2O), solvent B (0.1% TFA in acetonitrile), gradient 0% B to 60% B over 45 minutes, gradient 60% B to 100% B over 3 minutes, 100% B for 7 minutes, pre-run 0% B for 10 minutes, C18 Phenomenex Luna column (5 μm, 250×10 mm). Mass of glucagon-PDE was confirmed by the direct infusion of purified and dried HPLC fraction in ESI-MS in 50% v/v H2O/ACN (0.1% TFA) (FIG. 5B). ESI-MS (m/z): [M] calculated for glucagon-PDE, 3698.0; observed deconvoluted mass, 3697.0; See FIG. 6A and FIG. 6B for HPLC, MS analyses of glucagon-PDE.

Glucagon-CE Synthesis

Turning to FIG. 7, in an exemplary embodiment, 0.92 μmoles of glucagon-PDE in DMSO was combined with approximately 13.7 μmoles of CE peptide in DMSO. Purified and dried glucagon-PDE was reconstituted in DMSO and quantitated using UV/vis spectrophotometer. Final reaction volume was 0.218 mL. The reaction mixture was kept at room temperature overnight.

The reaction was analyzed for the formation of the product using reversed phase HPLC. Reversed phase HPLC (flow rate 0.4 mL/min, runtime: 30 minutes with 5 minutes post-run), solvent A (0.1% TFA in H2O), solvent B (0.1% TFA in acetonitrile), gradient 0% B to 100% B over 28 minutes, 100% B to 0% B over 2 minutes, post-run 0% B for 5 minutes, C18 Supelco Nucleosil column (5 μm, 150×3.2 mm). Glucagon-CE eluted at 17.120 minutes. Glucagon-CE was purified using semi preparative reversed phase HPLC. Reversed phase HPLC (flow rate 2 mL/min, runtime: 60 minutes with 10 minutes pre-run), solvent A (0.1% TFA in H2O), solvent B (0.1% TFA in acetonitrile), gradient 0% B to 60% B over 45 minutes, gradient 60% B to 100% B over 3 minutes, 100% B for 7 minutes, pre-run 0% B for 10 minutes, C18 Phenomenex Luna column (5 μm, 250×10 mm). Mass of glucagon-CE was confirmed by the direct infusion of purified and dried HPLC fraction in ESI-MS in 50% v/v H2O/ACN (0.1% TFA). ESI-MS (m/z): [M] calculated for glucagon-CE, 3878.1; observed deconvoluted mass, 3877.0; See FIG. 8A and FIG. 8B for HPLC, MS analyses of glucagon-CE.

Glucagon-CE2 Synthesis

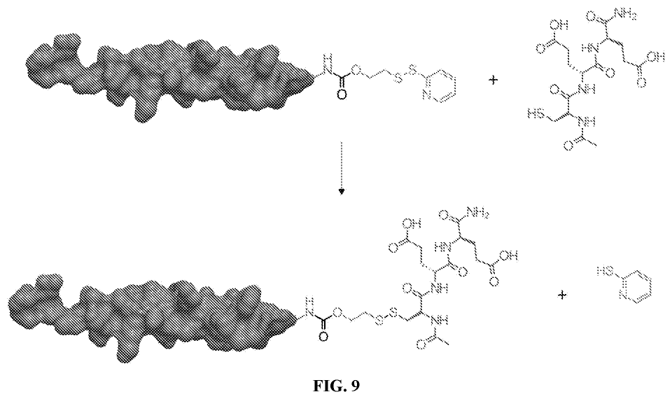

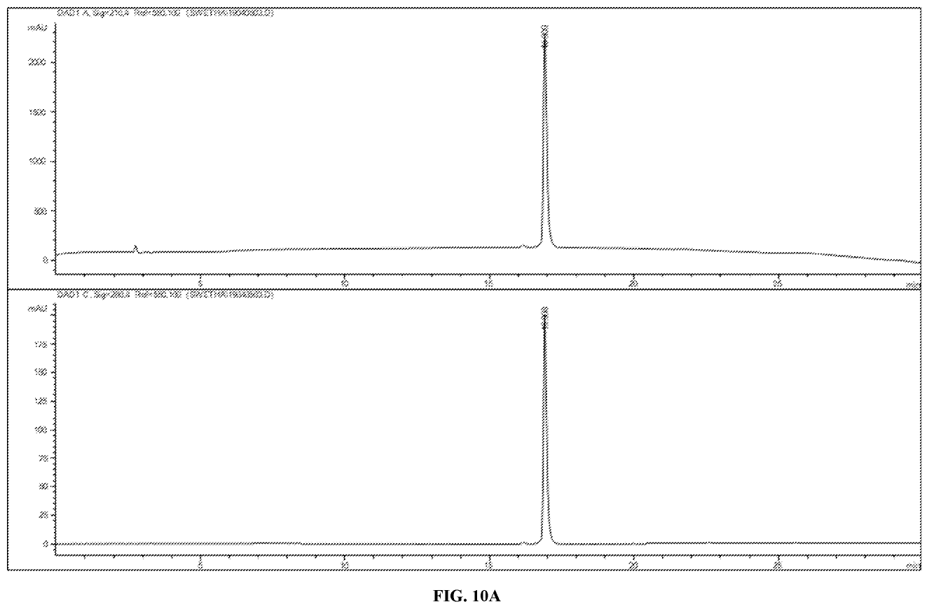

Turning to FIG. 9, in an exemplary embodiment, 0.92 μmoles of glucagon-PDE in DMSO was combined with approximately 13.7 μmoles of CE2 peptide in DMSO. Purified and dried glucagon-PDE was reconstituted in DMSO and quantitated using UV/vis spectrophotometer. Final reaction volume was 0.218 mL. The reaction mixture was kept at room temperature overnight.

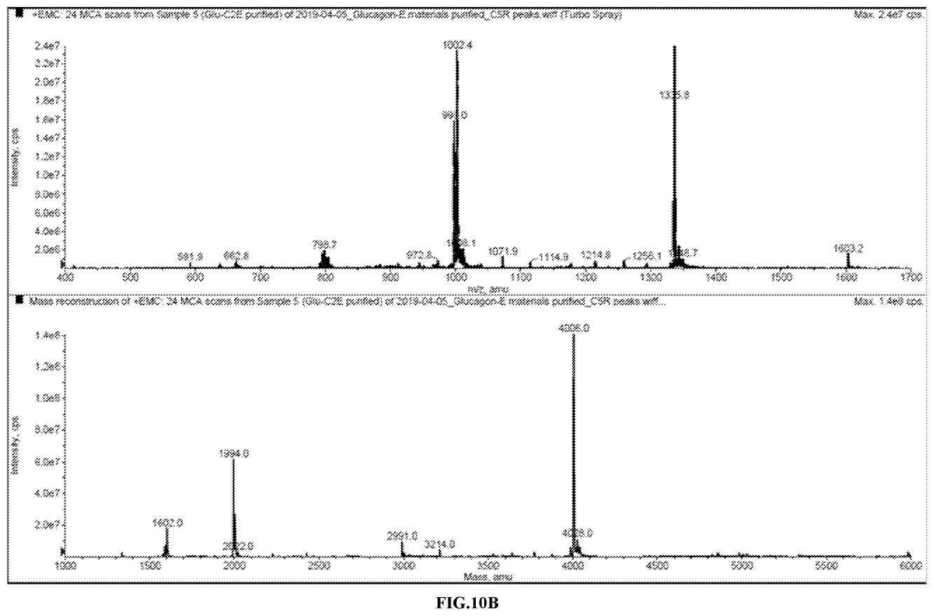

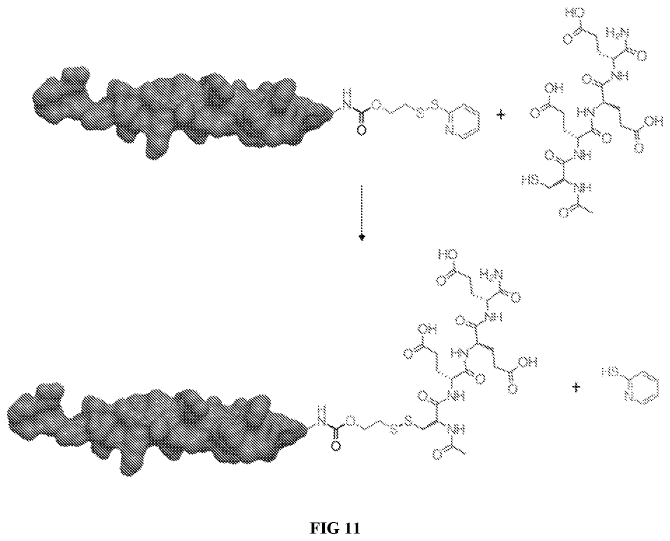

As depicted in FIGS. 10A and 10B, the reaction was analyzed for the formation of the product using reversed phase HPLC. Reversed phase HPLC (flow rate 0.4 mL/min, runtime: 30 minutes with 5 minutes post-run), solvent A (0.1% TFA in H2O), solvent B (0.1% TFA in acetonitrile), gradient 0% B to 100% B over 28 minutes, 100% B to 0% B over 2 minutes, post-run 0% B for 5 minutes, C18 Supelco Nucleosil column (5 μm, 150×3.2 mm). Glucagon-CE2 eluted at 16.908 minutes. Glucagon-CE2 was purified using semi preparative reversed phase HPLC. Reversed phase HPLC (flow rate 2 mL/min, runtime: 60 minutes with 10 minutes pre-run), solvent A (0.1% TFA in H2O), solvent B (0.1% TFA in acetonitrile), gradient 0% B to 60% B over 45 minutes, gradient 60% B to 100% B over 3 minutes, 100% B for 7 minutes, pre-run 0% B for 10 minutes, C18 Phenomenex Luna column (5 μm, 250×10 mm). Mass of glucagon-CE2 was confirmed by the direct infusion of purified and dried HPLC fraction in ESI-MS in 50% v/v H2O/ACN (0.1% TFA). ESI-MS (m/z): [M] calculated for glucagon-CE2, 4007.1; observed deconvoluted mass, 4006.0; See FIG. 10A and FIG. 10B for HPLC, MS analyses of glucagon-CE2.

Glucagon-CE3 Synthesis

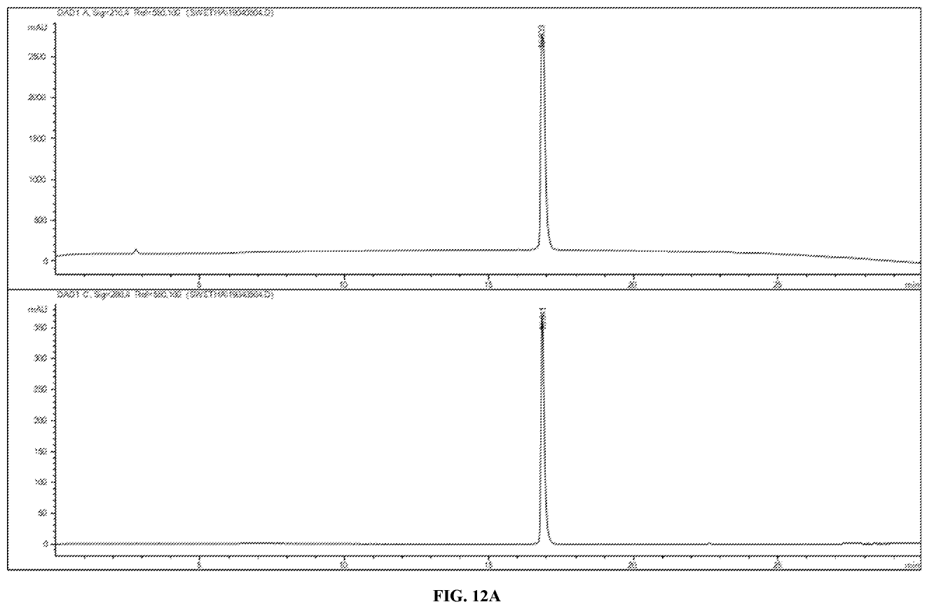

Turning to FIG. 11, in an exemplary embodiment, 0.92 μmoles of glucagon-PDE in DMSO was combined with approximately 13.7 μmoles of CE3 peptide in DMSO. Purified and dried glucagon-PDE was reconstituted in DMSO and quantitated using UV/vis spectrophotometer. Final reaction volume was 0.218 mL. The reaction mixture was kept at room temperature overnight.

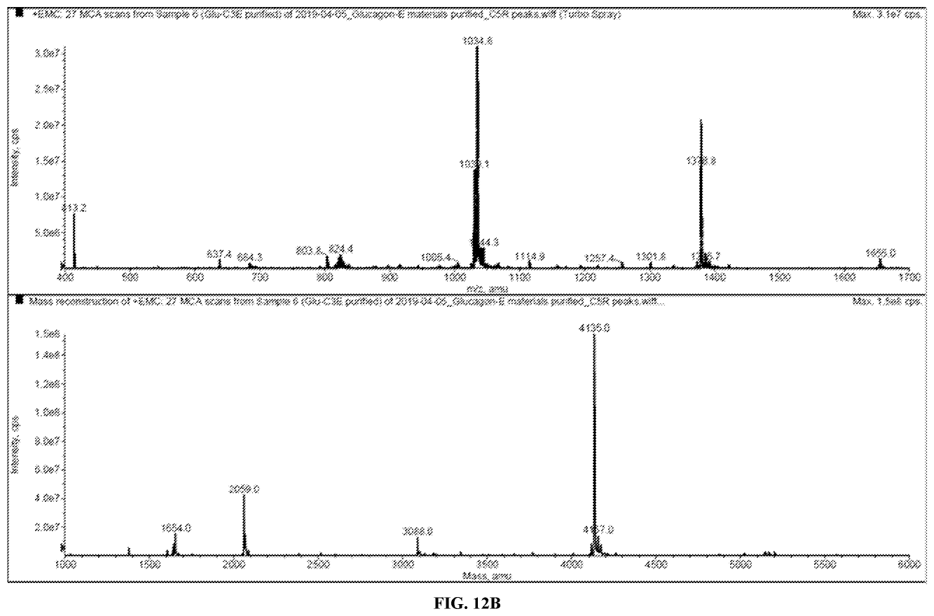

The reaction was analyzed for the formation of the product using reversed phase HPLC. Reversed phase HPLC (flow rate 0.4 mL/min, runtime: 30 minutes with 5 minutes post-run), solvent A (0.1% TFA in H2O), solvent B (0.1% TFA in acetonitrile), gradient 0% B to 100% B over 28 minutes, 100% B to 0% B over 2 minutes, post-run 0% B for 5 minutes, C18 Supelco Nucleosil column (5 μm, 150×3.2 mm). Glucagon-CE3 eluted at 16.841 minutes. Glucagon-CE3 purified using semi preparative reversed phase HPLC. Reversed phase HPLC (flow rate 2 mL/min, runtime: 60 minutes with 10 minutes pre-run), solvent A (0.1% TFA in H2O), solvent B (0.1% TFA in acetonitrile), gradient 0% B to 60% B over 45 minutes, gradient 60% B to 100% B over 3 minutes, 100% B for 7 minutes, pre-run 0% B for 10 minutes, C18 Phenomenex Luna column (5 μm, 250×10 mm). Mass of glucagon-CE3 was confirmed by the direct infusion of purified and dried HPLC fraction in ESI-MS in 50% v/v H2O/ACN (0.1% TFA). ESI-MS (m/z): [M] calculated for glucagon-CE3, 4136.2; observed deconvoluted mass, 4135.0; See FIG. 12A and FIG. 12B for HPLC, MS analyses of glucagon-CE3.

Glucagon-CE4 Synthesis

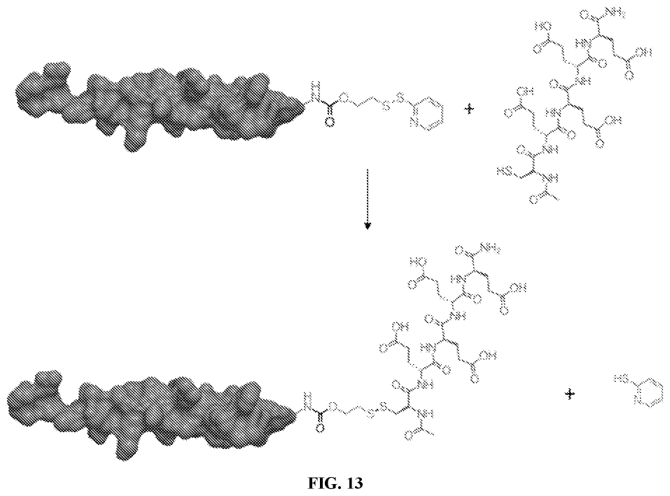

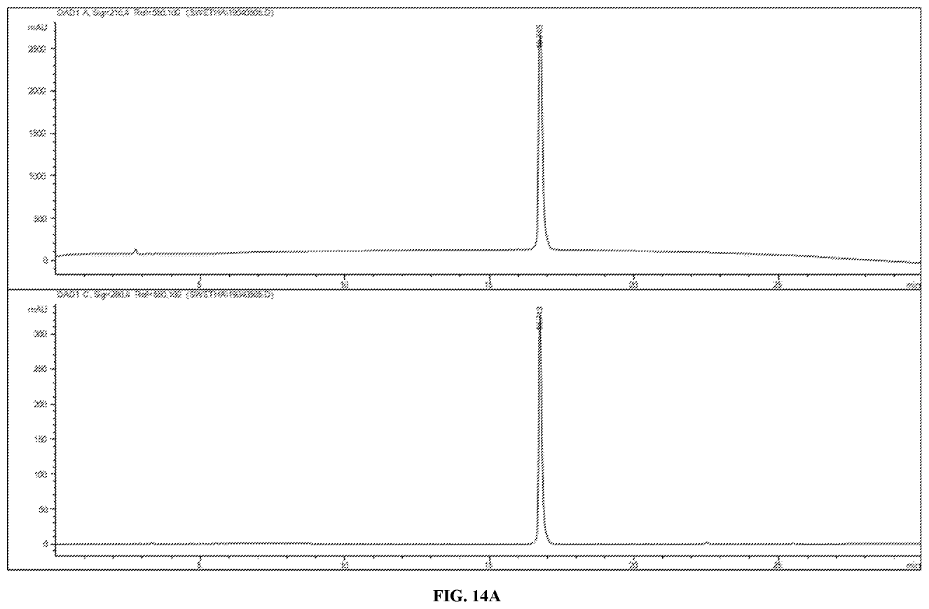

Turning to FIG. 13, in an exemplary embodiment, 0.95 μmoles of glucagon-PDE in DMSO was combined with approximately 14.25 μmoles of CE4 peptide in DMSO. Purified and dried glucagon-PDE was reconstituted in DMSO and quantitated using UV/vis spectrophotometer. Final reaction volume was 0.19 mL. The reaction mixture was kept at room temperature overnight.

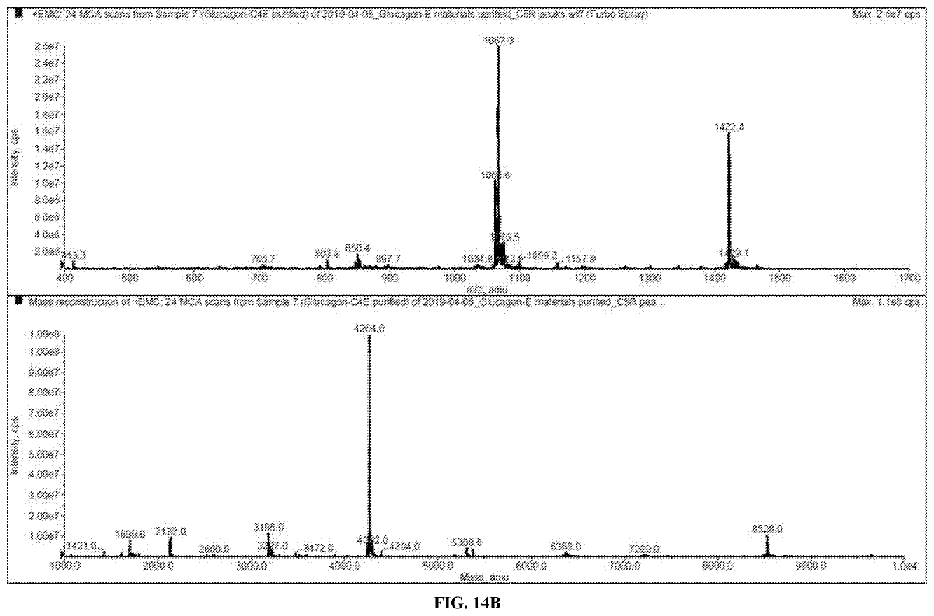

The reaction was analyzed for the formation of the product using reversed phase HPLC. Reversed phase HPLC (flow rate 0.4 mL/min, runtime: 30 minutes with 5 minutes post-run), solvent A (0.1% TFA in H2O), solvent B (0.1% TFA in acetonitrile), gradient 0% B to 100% B over 28 minutes, 100% B to 0% B over 2 minutes, post-run 0% B for 5 minutes, C18 Supelco Nucleosil column (5 μm, 150×3.2 mm). Glucagon-CE4 eluted at 16.743 minutes. Glucagon-CE4 was purified using semi preparative reversed phase HPLC. Reversed phase HPLC (flow rate 2 mL/min, runtime: 60 minutes with 10 minutes pre-run), solvent A (0.1% TFA in H2O), solvent B (0.1% TFA in acetonitrile), gradient 0% B to 60% B over 45 minutes, gradient 60% B to 100% B over 3 minutes, 100% B for 7 minutes, pre-run 0% B for 10 minutes, C18 Phenomenex Luna column (5 μm, 250×10 mm). Mass of glucagon-CE4 was confirmed by the direct infusion of purified and dried HPLC fraction in ESI-MS in 50% v/v H2O/ACN (0.1% TFA). ESI-MS (m/z): [M] calculated for glucagon-CE4, 4265.2; observed deconvoluted mass, 4264.0; See FIG. 14A and FIG. 14B for HPLC, MS analyses of glucagon-CE4.

Glucagon-CE5 Synthesis

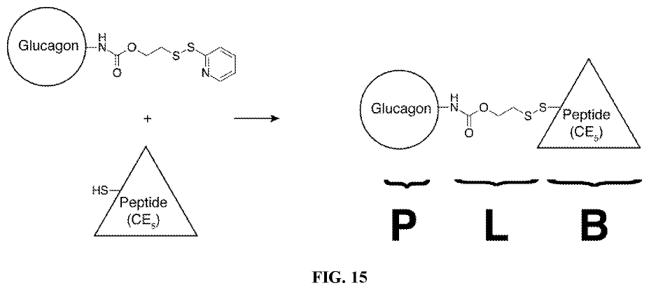

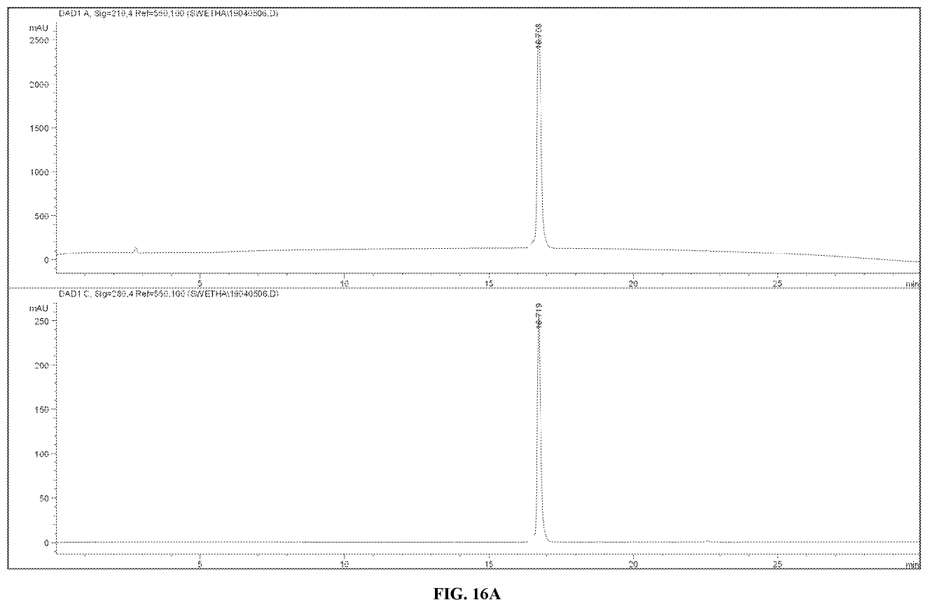

Turning to FIG. 15, in an exemplary embodiment, 0.95 μmoles of glucagon-PDE in DMSO was combined with approximately 14.25 μmoles of CE5 peptide in DMSO. Purified and dried glucagon-PDE was reconstituted in DMSO and quantitated using UV/vis spectrophotometer. Final reaction volume was 0.19 mL. The reaction mixture was kept at room temperature overnight.

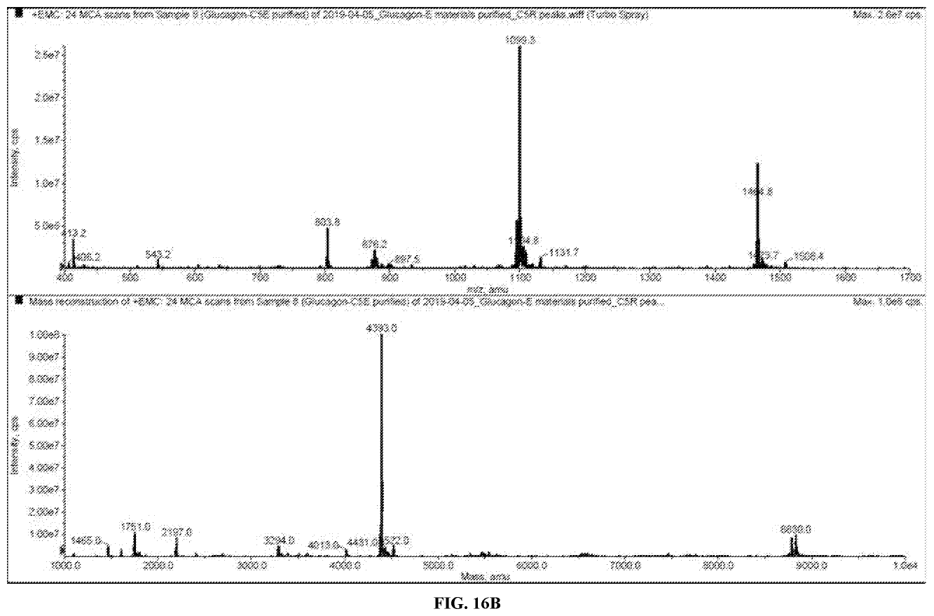

The reaction was analyzed for the formation of the product using reversed phase HPLC. Reversed phase HPLC (flow rate 0.4 mL/min, runtime: 30 minutes with 5 minutes post-run), solvent A (0.1% TFA in H2O), solvent B (0.1% TFA in acetonitrile), gradient 0% B to 100% B over 28 minutes, 100% B to 0% B over 2 minutes, post-run 0% B for 5 minutes, C18 Supelco Nucleosil column (5 μm, 150×3.2 mm). Glucagon-CE5 eluted at 18.719 minutes. Glucagon-CE5 was purified using semi preparative reversed phase HPLC. Reversed phase HPLC (flow rate 2 mL/min, runtime: 60 minutes with 10 minutes pre-run), solvent A (0.1% TFA in H2O), solvent B (0.1% TFA in acetonitrile), gradient 0% B to 60% B over 45 minutes, gradient 60% B to 100% B over 3 minutes, 100% B for 7 minutes, pre-run 0% B for 10 minutes, C18 Phenomenex Luna column (5 μm, 250×10 mm). Mass of glucagon-CE5 was confirmed by the direct infusion of purified and dried HPLC fraction in ESI-MS in 50% v/v H2O/ACN (0.1% TFA). ESI-MS (m/z): [M] calculated for glucagon-CE5, 4394.3; observed deconvoluted mass, 4393.0; See FIG. 16A and FIG. 16B for HPLC, MS analyses of glucagon-CE5.

From the foregoing it will be seen that this invention is one well adapted to attain all ends and objectives herein-above set forth, together with the other advantages which are obvious and which are inherent to the invention.

Since many possible embodiments may be made of the invention without departing from the scope thereof, it is to be understood that all matters herein set forth or shown in the accompanying drawings are to be interpreted as illustrative, and not in a limiting sense.

While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

It is noted that, as used in this specification and the appended claims, the singular forms “a,” “an,” and “the” include plural referents unless expressly and unequivocally limited to one referent. Thus, for example, reference to “a molecule” includes molecules.

We claim:

1. A method for reversibly preventing a protein from interacting with a second protein comprising:
providing the protein;
providing a blocking group;
providing a linking moiety; and
forming a protein blocking assembly by forming a first bond between the linking moiety and the protein and forming a second bond between the linking moiety and the blocking group;
wherein the blocking group comprises a chemical structure that prevents interaction with the second protein due to steric or static interference; and
wherein at least one of the first bond and the second bond is cleavable.

2. The method of claim 1, wherein the protein blocking assembly inhibits formation of protein-protein complexes.

3. The method of claim 1, wherein the protein-protein complex is selected from the group consisting of fibrils and hexamers.

4. The method of claim 1, wherein the stearic interference is caused by the blocking group's size, shape or combinations thereof.

5. The method of claim 1, wherein the electrostatic interference is caused by the blocking group's charge, partial charge or combinations thereof.

6. The method of claim 1, wherein the blocking group comprises a peptide, lipid, small molecule, nucleic acid, saccharide or combinations thereof.

7. The method of claim 1, wherein the blocking group comprises the peptide, and wherein the peptide comprises a sequence of CEₙ, where n=1-5, preferably n=5.

8. The method of claim 1, wherein the protein is selected from the group consisting of insulin, glucagon, immunoglobulin, their derivatives or analogs, and combinations thereof.

9. The method of claim 1, wherein the first bond to the protein is via a side chain functional group of the protein.

10. The method of claim 9, wherein the side chain functional group is of the protein selected from the group consisting of amine, alcohol, carboxylic acid, guandinium, amide, thiol and combinations thereof.

11. The method of claim 1, wherein at least one of the first bond and the second bond is cleavable by chemical effector or enzyme.

12. The method of claim 11, wherein the chemical effector or enzyme is endogenous in a human or animal body.

13. The method of claim 1, wherein at least one of the first bond and the second bond is cleavable by hydrolysis by esterases, hydrolysis by peptidases, hydrolysis by phosphatases, hydrolysis by other enzymes, reduction, oxidation, or combinations thereof.

14. The method of claim 1, wherein the linker moiety comprises activated pyridyl dithio ethanol (PDE).

15. The method of claim 14, wherein the activated PDE is activated with carbonyldiimidazole (CDI).

16. The method of claim 1, wherein at least one of the first and second bond comprises one or more esters, amides, carbamates, carbonates, phospho-esters, phosphor-amides, di-sulfides, ethers, ketals, aminals, acetals, sulfonamides, imines, hydrazones, or combinations thereof.

17. The protein blocking assembly produced by the method of claim 1.

18. A method of using the protein blocking assembly of claim 17 comprising:
administering the protein blocking assembly to a human or animal in need of the protein;
wherein at least one of the first bond and second bond is cleaved within the human or animal; and
wherein the cleavage releases the protein from the protein blocking assembly.

19. The method of claim 18, wherein cleavage occurs by a chemical or biochemical processes endogenous to the human or animal.

20. A protein blocking assembly comprising:
a protein;
a blocking group;
a linking moiety reversibly linking the protein and the blocking group,
wherein the blocking group comprises a chemical structure that prevents interaction with a second protein due to steric or static interference.

21. The protein blocking assembly of claim 20, wherein the protein blocking assembly inhibits formation of protein-protein complexes.

22. The protein blocking assembly of claim 21, wherein the protein-protein complex is selected from the group consisting of fibrils and hexamers.

23. The protein blocking assembly of claim 20, wherein the stearic interference is caused by the blocking group's size, shape or combinations thereof.

24. The protein blocking assembly of claim 20, wherein the electrostatic interference is caused by the blocking group's charge, partial charge or combinations thereof.

25. The protein blocking assembly of claim 20, wherein the blocking group comprises a peptide, lipid, small molecule, nucleic acid, saccharide or combinations thereof.

26. The protein blocking assembly of claim 20, wherein the blocking group comprises the peptide, and wherein the peptide comprises a sequence of CEₙ, where n=1-5, preferably n=5.

27. The protein blocking assembly of claim 20, wherein the protein is selected from the group consisting of insulin, glucagon, immunoglobulin, their derivatives or analogs, and combinations thereof.

28. The protein blocking assembly of claim 20, wherein a bond between the linking moiety and the protein is via a side chain functional group of the protein.

29. The protein blocking assembly of claim 28, wherein the side chain functional group is of the protein selected from the group consisting of amine, alcohol, carboxylic acid, guandinium, amide, thiol and combinations thereof.

30. The protein blocking assembly of claim 20, wherein a bond between the linking moiety and the protein is cleavable by chemical effector or enzyme.

31. The protein blocking assembly of claim 30, wherein the chemical effector or enzyme is endogenous in a human or animal body.

32. The protein blocking assembly of claim 20, wherein a bond between the linking moiety and the protein is cleavable by hydrolysis by esterases, hydrolysis by peptidases, hydrolysis by phosphatases, hydrolysis by other enzymes, reduction, oxidation, or combinations thereof.

33. The protein blocking assembly of claim 20, wherein the linker moiety comprises activated pyridyl dithio ethanol (PDE).

34. The protein blocking assembly of claim 33, wherein the activated PDE is activated with carbonyldiimidazole (CDI).

35. The protein blocking assembly of claim 20, wherein a bond between the linking moiety and the protein comprises one or more esters, amides, carbamates, carbonates, phospho-esters, phosphor-amides, di-sulfides, ethers, ketals, aminals, acetals, sulfonamides, imines, hydrazones, or combinations thereof.

36. A method for preventing a protein from interacting with a second protein comprising:
providing the protein;
providing a blocking group;
providing a linking moiety; and
forming a protein blocking assembly by forming a first bond between the linking moiety and the protein and forming a second bond between the linking moiety and the blocking group;
wherein the blocking group comprises a chemical structure that prevents interaction with the second protein due to steric or static interference; and
wherein at least one of the first bond and the second bond is cleavable.

37. The method of claim 36, wherein the protein blocking assembly inhibits formation of protein-protein complexes.

38. The method of claim 37, wherein the protein-protein complex is selected from the group consisting of fibrils and hexamers.

39. The method of any of claims 36 to 38, wherein the stearic interference is caused by the blocking group's size, shape or combinations thereof.

40. The method of any of claims 36 to 39, wherein the electrostatic interference is caused by the blocking group's charge, partial charge or combinations thereof.

41. The method of any of claims 36 to 40, wherein the blocking group comprises a peptide, lipid, small molecule, nucleic acid, saccharide or combinations thereof.

42. The method of any of claims 36 to 41, wherein the blocking group comprises the peptide, and wherein the peptide comprises a sequence of CEₙ, where n=1-5, preferably n=5.

43. The method of any of claims 36 to 42, wherein the protein is selected from the group consisting of insulin, glucagon, immunoglobulin, their derivatives or analogs, and combinations thereof.

44. The method of any of claims 36 to 43, wherein the first bond to the protein is via a side chain functional group of the protein.

45. The method of claim 44, wherein the side chain functional group is of the protein selected from the group consisting of amine, alcohol, carboxylic acid, guandinium, amide, thiol and combinations thereof.

46. The method of any of claims 36 to 45, wherein at least one of the first bond and the second bond is cleavable by chemical effector or enzyme.

47. The method of claim 46, wherein the chemical effector or enzyme is endogenous in a human or animal body.

48. The method of any of claims 36 to 47, wherein at least one of the first bond and the second bond is cleavable by hydrolysis by esterases, hydrolysis by peptidases, hydrolysis by phosphatases, hydrolysis by other enzymes, reduction, oxidation, or combinations thereof.

49. The method of any of claims 36 to 48, wherein the linker moiety comprises activated pyridyl dithio ethanol (PDE).

50. The method of claim 49, wherein the activated PDE is activated with carbonyldiimidazole (CDI).

51. The method of any of claims 36 to 50, wherein at least one of the first and second bond comprises one or more esters, amides, carbamates, carbonates, phospho-esters, phosphor-amides, di-sulfides, ethers, ketals, aminals, acetals, sulfonamides, imines, hydrazones, or combinations thereof.

52. The protein blocking assembly produced by the method of any of claims 36 to 51.

53. A method of using the protein blocking assembly of claim 52 comprising:
administering the protein blocking assembly to a human or animal in need of the protein;
wherein at least one of the first bond and second bond is cleaved within the human or animal; and
wherein the cleavage releases the protein from the protein blocking assembly.

54. The method of claim 53, wherein cleavage occurs by a chemical or biochemical processes endogenous to the human or animal.

55. A protein blocking assembly comprising:
a protein;
a blocking group;
a linking moiety reversibly linking the protein and the blocking group,
wherein the blocking group comprises a chemical structure that prevents interaction with a second protein due to steric or static interference.

56. The protein blocking assembly of claim 55, wherein the protein blocking assembly inhibits formation of protein-protein complexes.

57. The protein blocking assembly of claim 56, wherein the protein-protein complex is selected from the group consisting of fibrils and hexamers.

58. The protein blocking assembly of any of claims 55 to 57, wherein the stearic interference is caused by the blocking group's size, shape or combinations thereof.

59. The protein blocking assembly of any of claims 55 to 58, wherein the electrostatic interference is caused by the blocking group's charge, partial charge or combinations thereof.

60. The protein blocking assembly of any of claims 55 to 59, wherein the blocking group comprises a peptide, lipid, small molecule, nucleic acid, saccharide or combinations thereof.

61. The protein blocking assembly of any of claims 55 to 60, wherein the blocking group comprises the peptide, and wherein the peptide comprises a sequence of CEₙ, where n=1-5, preferably n=5.

62. The protein blocking assembly of any of claims 55 to 61, wherein the protein is selected from the group consisting of insulin, glucagon, immunoglobulin, their derivatives or analogs, and combinations thereof.

63. The protein blocking assembly of any of claims 55 to 62, wherein a bond between the protein and the linking moiety is via a side chain functional group of the protein.

64. The protein blocking assembly of claim 63, wherein the side chain functional group is of the protein selected from the group consisting of amine, alcohol, carboxylic acid, guandinium, amide, thiol and combinations thereof.

65. The protein blocking assembly of any of claims 55 to 64, wherein a bond between the protein and the linking moiety is cleavable by chemical effector or enzyme.

66. The protein blocking assembly of claim 65, wherein the chemical effector or enzyme is endogenous in a human or animal body.

67. The protein blocking assembly of any of claims 55 to 66, wherein a bond between the protein and the linking moiety is cleavable by hydrolysis by esterases, hydrolysis by peptidases, hydrolysis by phosphatases, hydrolysis by other enzymes, reduction, oxidation, or combinations thereof.

68. The protein blocking assembly of any of claims 55 to 67, wherein the linker moiety comprises activated pyridyl dithio ethanol (PDE).

69. The protein blocking assembly of claim 68, wherein the activated PDE is activated with carbonyldiimidazole (CDI).

70. The protein blocking assembly of any of claims 55 to 69, wherein a bond between the protein and the linking moiety comprises one or more esters, amides, carbamates, carbonates, phospho-esters, phosphor-amides, di-sulfides, ethers, ketals, aminals, acetals, sulfonamides, imines, hydrazones, or combinations thereof.
